(19) United States
(12) Patent Application Publication
(10) Pub. No.: US 2004/0190795 A1
(43) Pub. Date: Sep. 30, 2004

(54) Image sending device and image receiving device

(75) Inventor: Takeshi Funahashi, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/811,387

(22) Filed: Mar. 29, 2004

(30) Foreign Application Priority Data
Mar. 27, 2003 (JP) 087891/2003

(51) Int. Cl. B41J 1/00; B41B 1/00; B41F 1/00; H04L 9/00; G06F 12/14; G06K 9/60; A61B 8/06

(52) U.S. Cl. 382/307; 358/1.15; 713/150

(57) ABSTRACT

A medical image is separated into an image body M1 and supplementary information M2 including patient privacy information. The supplementary information M2 is sent using a high security dedicated circuit 4 and the image body M1 is sent using a public circuit 3. Since the data volume of the supplementary information M2 is less than the data volume of the image body M1, communication costs for using the dedicated circuit 4 can be reduced compared to a case of sending and receiving a whole medical image.

### BACKGROUND OF THE INVENTION

1. Field of the Invention

The present invention relates to an image sending device for sending a medical image to an external device and an image receiving device for receiving the medical image which has been sent by the image sending device.

2. Description of the Related Art

Conventionally, systems are used in the field of medical imaging, wherein a medical image such as a radiographic image of a patient is stored in an image server in a hospital, and the medical image is sent to and displayed at a terminal device such as a personal computer owned by a doctor according to a request from the terminal device. Since the doctor can refer to a required image at the terminal device by storing the medical image in advance in the image server, which has been obtained by photographing the patient using input modalities such as a CR (Computed Radiography) device, a CT (Computer Tomography) device, and a MRI (Magnetic Resonance Imaging) device, the doctor can efficiently diagnose the patient in these systems.

Further, in addition to sending the medical image from the terminal device of the doctor to the image server, doctors also exchange medical images directly among themselves in some cases.

Here, patient information which is patient personal information such as the patient name, ID, sex, and date of birth and patient privacy information (hereinafter called privacy information) such as findings by a radiologist who has observed the medical image and examination information including an examination result, an examination date, or the like are included in supplementary information and attached to the medical image. Therefore, a system has been proposed, wherein the medical image is sent and received using a high security dedicated circuit to prevent leakage of the supplementary information in the case that the medical image is sent and received between the terminal devices or between the terminal device and the image server (refer to Japanese Unexamined Patent Publication No. 2002-269243). Further, the medical image is encrypted using a sophisticated encryption method so that the encrypted medical image cannot easily be decrypted, or the medical image is sent and received using a VPN (Virtual Private Network) by encrypting a communication packet and concealing a communication destination.

However, usage charges for the dedicated circuit are high, and a long communication time is required for sending and receiving large volume data such as the medical image. Consequently, there is a problem that communication costs for sending and receiving the medical image becomes high. Further, if the volume of data is large, such as in the case of the medical image, a long time is also required for performing encryption. Further, since high performance equipment is required to perform sophisticated encryption, there is also a problem that equipment costs become high.

### SUMMARY OF THE INVENTION

In view of the foregoing circumstances, it is an object of the present invention to send and receive medical images, to which the patient privacy information is attached as supplementary information, at low cost while maintaining high security.

An image sending device according to the present invention, which has a sending means for sending a medical image, to which supplementary information including patient privacy information has been attached, to an external device includes: a separating means for separating the medical image into the supplementary information and an image body, and a transmission control means for controlling the sending means so that the sending means sends the supplementary information using a higher security transmission method compared to a transmission method for sending the image body.

The “patient privacy information” is patient information which is the patient personal information such as the patient name, ID, sex, and date of birth, findings by a radiologist who has observed the medical image, and examination information such as an examination result, an examination date, or the like. The supplementary information is represented by characters, and the data volume of the supplementary information is very small compared to the data volume of the image body.

Further, the high security transmission method may also be a transmission method for encrypting the supplementary information using a more sophisticated encryption method compared to an encryption method for encrypting the image body, in the image sending device according to the present invention.

The “more sophisticated encryption method” is an encryption method which makes it more difficult to decrypt the supplementary information by encrypting the supplementary information through more complex processing than an encryption method for the medical image, from which the supplementary information has been separated. A stream encryption method, a block encryption method, and an encryption method using MAC (Message Authentication Code), which are used in communication protocols such as the Internet, may be used as the encryption method.

Further, the high security transmission method may also be a transmission method for sending the supplementary information using a higher security communication circuit such as the dedicated circuit, compared to a communication circuit for sending the image body, in the image sending device according to the present invention.

Further, the image sending device according to the present invention may also include a selection accepting means for accepting a selection of a first transmission method for encrypting the supplementary information using the more sophisticated encryption method compared to the encryption method for encrypting the image body and/or a second transmission method for sending the supplementary information using the higher security communication circuit compared to the communication circuit for sending the image body as the high security transmission method. The transmission control means may also control the sending means so that the sending means sends the supplementary information using the transmission method which has been accepted by the selection accepting means.

An image receiving device according to the present invention includes a receiving means for receiving the supplementary information and the image body which have been sent by the image sending means according to the present invention and an associating means for associating the supplementary information with the image body.

Another image sending device according to the present invention, which has the sending means for sending the medical image, to which the supplementary information including the patient privacy information has been attached, to the external device includes a separating means for separating the medical image into the supplementary information and the image body, and a transmission control means for controlling the sending means so that the sending means sends only the image body.

The other image sending-device according to the present invention may further include a recording means for recording the supplementary information in a recording medium such as FD, CD-R, and MO. Further, the other image sending device according to the present invention may also include a print-out means for printing out the supplementary information.

According to the present invention, the separating means separates the medical image into the supplementary information and the image body, and the transmission control means controls the sending means so that the sending means sends the supplementary information using the higher security transmission method compared to the transmission method for sending the image body. Here, a communication cost is high, and a long time is required for performing encryption processing or the like in the high security transmission method. However, since the volume of the supplementary information is small compared to the volume of the medical image, a time required for communication and a time required for encryption processing can be reduced compared to the case of sending the medical image as is or encrypting the medical image as is. Consequently, the communication and processing costs can be reduced. Therefore, the medical image can be sent and received at low cost while high security is maintained.

If the high security transmission method is a transmission method for encrypting the supplementary information using a sophisticated encryption method compared to an encryption method for encrypting the image body, the medical image can be sent and received without using a communication circuit with a high communication cost, such as the dedicated circuit, while high security is maintained.

Further, if the high security transmission method is a transmission method for sending the supplementary information using a higher security communication circuit compared to a communication circuit for sending the image body, a processing environment using a high performance modem or a high performance router is not required to prevent a deterioration in data transfer performance in the case of encryption. Therefore, the medical image can be sent and received without a large investment in equipment costs, while high security is maintained.

Further, if the separating means separates the medical image into the supplementary information and the image body and the transmission control means controls the sending means so that the sending means sends only the image body, leakage of the patient privacy information can be prevented.

In this case, a recording medium can be delivered to a transmission destination of the image body, by recording the supplementary information in the recording medium, or printed-out supplementary information can be delivered or sent by facsimile to the transmission destination of the image body, by printing out the supplementary information. Accordingly, the image body can easily be associated with the supplementary information at the transmission destination of the image body.

### BRIEF DESCRIPTION OF THE DRAWINGS

FIG. 1 is a schematic block diagram showing the configuration of a medical image sending and receiving system to which an image sending device and an image receiving device according to a first embodiment of the present invention have been applied;

FIG. 2 is a schematic block diagram showing the configuration of a personal computer;

FIGS. 3A and 3B show a file structure of a medical image;

FIG. 4 shows a flow chart of processing performed at the time of sending the medical image from the personal computer in the first embodiment;

FIG. 5 shows a flow chart of processing performed at the time of receiving the medical image by the personal computer in the first embodiment;

FIG. 6 is a schematic block diagram showing the configuration of a medical image sending and receiving system to which an image sending device and an image receiving device according to a second embodiment of the present invention have been applied;

FIG. 7 shows a flow chart of processing performed at the time of sending the medical image from the personal computer in the second embodiment;

FIG. 8 shows a flow chart of processing performed at the time of receiving the medical image by the personal computer in the second embodiment;

FIG. 9 shows a flow chart of processing performed at the time of sending the medical image from the personal computer in a third embodiment;

FIG. 10 shows a flow chart of processing performed at the time of receiving the medical image by the personal computer in the third embodiment;

FIG. 11 shows a flow chart of processing performed at the time of sending the medical image from the personal computer in a fourth embodiment;

FIG. 12 shows a flow chart of processing performed at the time of receiving the medical image by the personal computer in the fourth embodiment; and

FIG. 13 is a schematic block diagram showing the configuration of a medical image sending and receiving system to which an image sending device and an image receiving device according to a fifth embodiment of the present invention have been applied.

### DESCRIPTION OF THE PREFERRED EMBODIMENTS

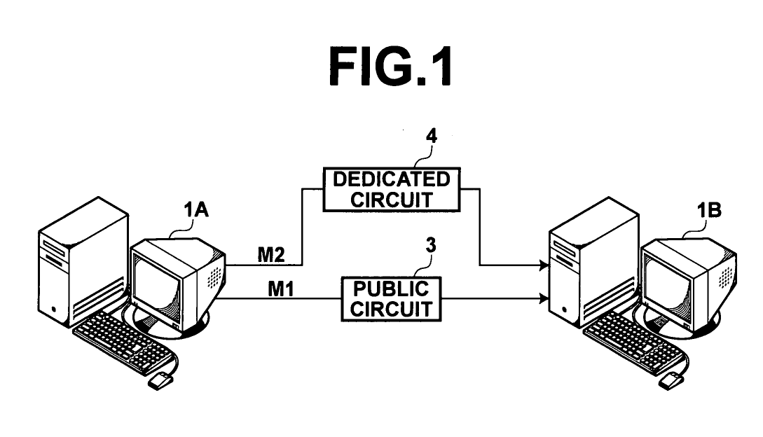

Hereinafter, embodiments of the present invention will be described with reference to the attached drawings. FIG. 1 is a schematic block diagram showing the configuration of a medical image sending and receiving system to which an image sending device and an image receiving device according to a first embodiment of the present invention have been applied. As illustrated in FIG. 1, the medical image sending and receiving system according to the present embodiment is a system where a medical image is sent and received between two personal computers 1A and 1B. The system is configured by connecting the personal computers 1A and 1B using a public circuit 3 and a dedicated circuit 4, such as telephone circuits.

Both of the personal computers 1A and 1B can send and receive the medical image. However, in the description of the present embodiment, it is assumed that the medical image is sent from the personal computer 1A to the personal computer 1B. Therefore, the medical image has been sent from a plurality of input modalities (not illustrated) such as a CR (Computed Radiography) device, a CT (Computer Tomography) device, and a MRI (Magnetic Resonance Imaging) device, and stored in the personal computer 1A.

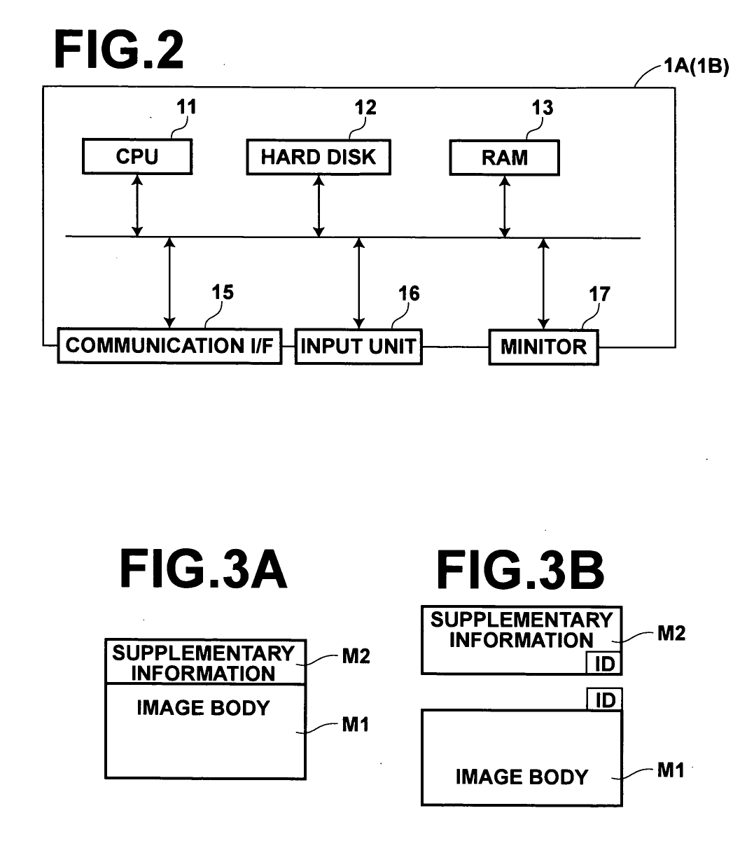

FIG. 2 is a schematic block diagram showing the configuration of the personal computer. Since configurations of the personal computers 1A and 1B are identical, only the configuration of the personal computer 1A will be described here. As illustrated in FIG. 2, the personal computer 1A includes a CPU 11 which controls operations of the personal computer 1A, a hard disk 12 which is a storing means for storing an operation program of the CPU 11, including a program for causing the personal computer 1A to operate as the image sending device and the image receiving device, a program for performing communication and various information such as the medical image, a RAM 13 which will become a work area during execution of the program, a communication interface 15 for connecting the personal computer 1A to the public circuit 3 and the dedicated circuit 4, an input unit 16 including a keyboard, a mouse, or the like for performing various inputs, and a monitor 17 for displaying various information.

The CPU 11 functions as a separating means, a transmission control means, and an associating means by reading the program for the image sending device and the image receiving device from the hard disk 12 and executing the program. Further, the CPU 11 and the communication interface 15 function as a sending means and a receiving means.

FIG. 3A shows a file structure of the medical image. As illustrated in FIG. 3A, the medical image includes an image body M1 and supplementary information M2 including patient information which is patient personal information such as the patient name, ID, sex, and date of birth and patient privacy information such as findings by a radiologist who has observed the medical image and examination information including an examination result, an examination date, or the like.

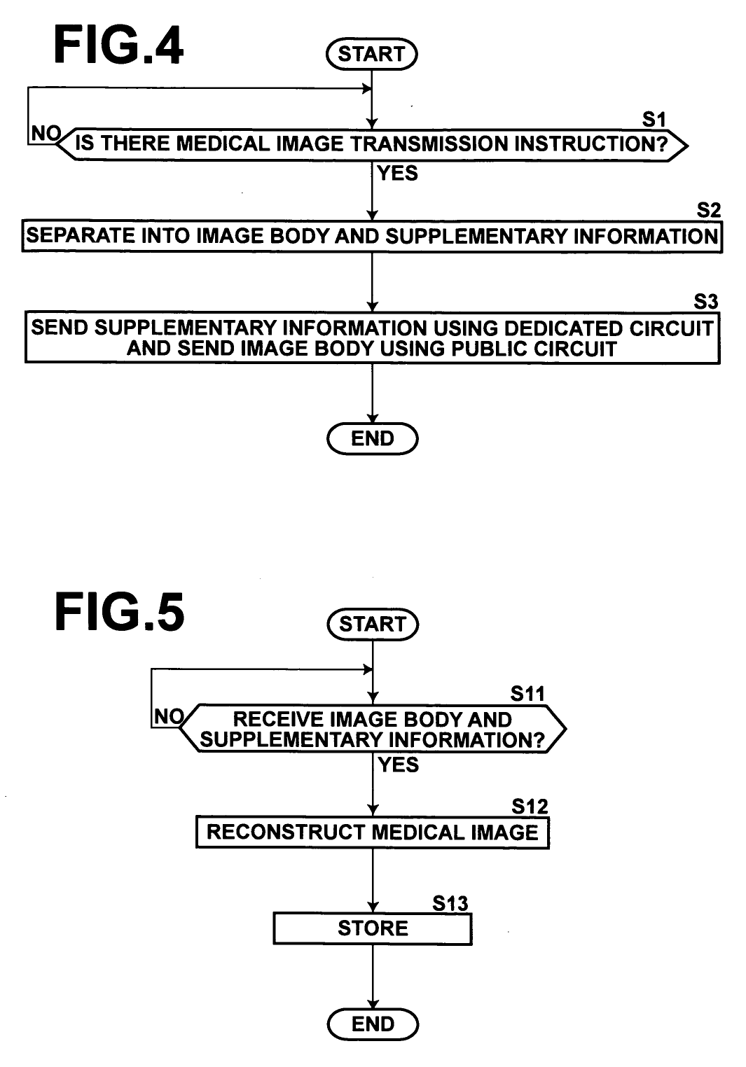

Next, processing performed in the first embodiment will be described. FIG. 4 shows a flowchart of processing performed at the time of sending the medical image from the personal computer 1A in the first embodiment. It is assumed that the medical image is stored in the hard disk 12 in the personal computer 1A. First, the CPU 11 in the personal computer 1A monitors whether a doctor who owns the personal computer 1A has given an instruction to send the medical image by using the input unit 16 (step S1). When step S1 is YES, the medical image, for which transmission has been instructed is separated into the image body M1 and the supplementary information, as illustrated in FIG. 3B (step S2). In this case, the patient ID included in the supplementary information M2 is copied and attached to the image body M1.

Then, the supplementary information M2 is sent to the personal computer 1B using the dedicated circuit 4, the image body M1 is sent to the personal computer 1B using the public circuit 3 (step S3), and the processing ends.

FIG. 5 shows a flow chart of processing performed at the time of receiving the medical image by the personal computer 1B in the first embodiment. First, the CPU 11 in the personal computer 1B monitors whether the image body M1 and the supplementary information M2 have been received (step S11). When step S11 is YES, the CPU 11 reconstructs the medical image by attaching the received supplementary information M2 to the image body M1, to which an ID corresponding to the ID included in the supplementary information M2 has been attached (step S12), and stores the reconstructed medical image in the hard disk 12 (step S13), and the processing ends.

The image body M1 and the supplementary information M2 may also be stored in the hard disk 12 by being associated with each other according to the ID instead of reconstructing the medical image. Specifically, the image body M1 and the supplementary information M2 should be linked by the ID.

Here, since the volume of the supplementary information M2 is small compared to the volume of the image body M1, the time required for communication can be reduced compared to the case of sending and receiving the medical image as is. Consequently, even if a circuit such as the dedicated circuit 4, for which usage charges are expensive, is used, the communication cost can be reduced compared to the case of sending and receiving the medical image as is. Therefore, the medical image can be sent and received at low cost while high security is maintained. Further, since a processing environment using a high performance modem or a high performance router such as that used in the case of encryption is not required to prevent a deterioration in data transfer performance, the medical image can be sent and received without a large investment in equipment costs, while security is maintained.

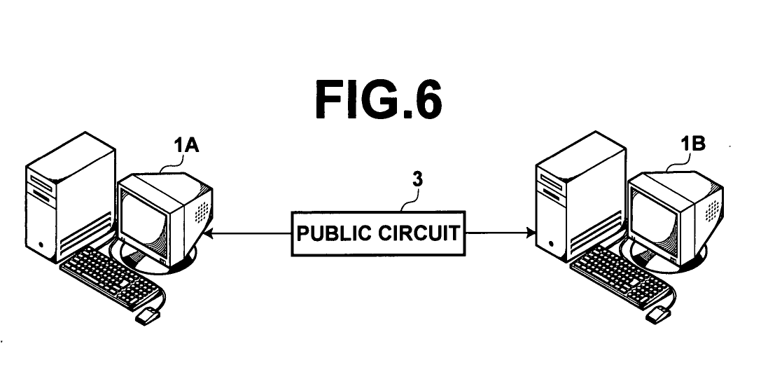

Next, a second embodiment of the present invention will be described. FIG. 6 is a schematic block diagram showing the configuration of a medical image sending and receiving system to which an image sending device and an image receiving device according to the second embodiment of the present invention have been applied. As illustrated in FIG. 6, the medical image sending and receiving system according to the present invention is a system where the medical image is sent and received between the two personal computers 1A and 1B. The system is configured by connecting the personal computers 1A and 1B using the public circuit 3.

The second embodiment differs from the first embodiment in that the medical image is separated into the image body M1 and the supplementary information M2, and the supplementary information M2 is encrypted using a higher security encryption method compared to an encryption method for encrypting the image body M1 and sent from the personal computer 1A to the personal computer 1B.

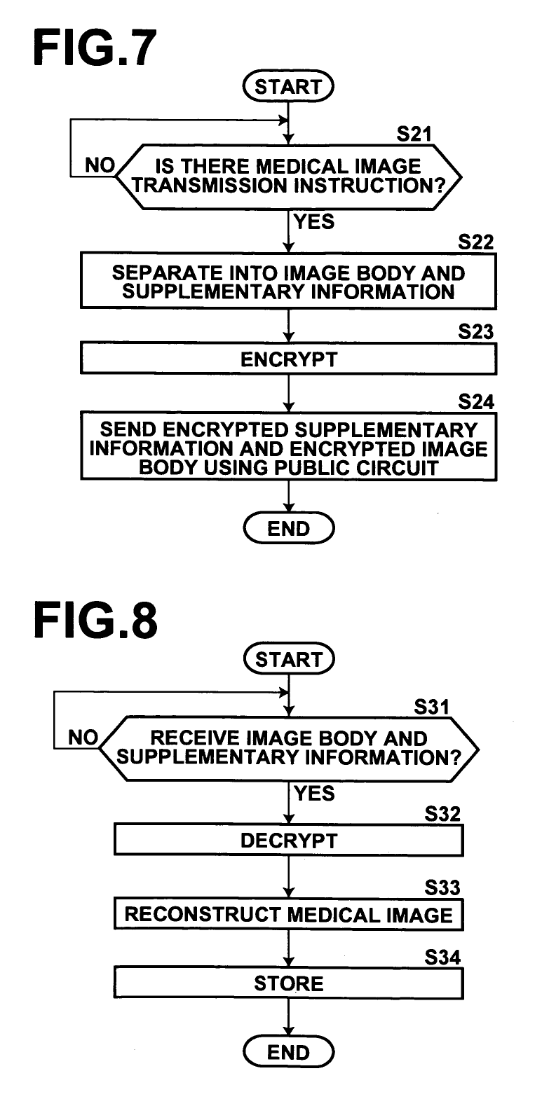

Next, processing performed in the second embodiment will be described. FIG. 7 shows a flowchart of processing performed at the time of sending the medical image from the personal computer 1A in the second embodiment. It is assumed that the medical image is stored in the hard disk 12 in the personal computer 1A. First, the CPU 11 in the personal computer 1A monitors whether the doctor who owns the personal computer 1A has given an instruction to send the medical image by using the input unit 16 (step S21). When step S21 is YES, the medical image, for which transmission has been instructed, is separated into the image body M1 and the supplementary information M2, as illustrated in FIG. 3B (step S22). In this case, the patient ID included in the supplementary information M2 is copied and attached to-the image body M1.

Then, the image body M1 and the supplementary information M2 are encrypted (step S23). In this case, the supplementary information M2 is encrypted using a sophisticated encryption method compared to an encryption method for encrypting the image body M1 so that the encrypted supplementary information M2 cannot easily be decrypted.

Then, the encrypted supplementary information M2 and the encrypted image body M1 are sent to the personal computer 1B using the public circuit 3 (step S24), and the processing ends.

FIG. 8 shows a flow chart of processing performed at the time of receiving the medical image by the personal computer 1B in the second embodiment. First, the CPU 11 in the personal computer 1B monitors whether the encrypted image body M1 and the encrypted supplementary information M2 have been received (step S31). When step S31 is YES, the CPU 11 decrypts the received image body M1 and the received supplementary information M2 (step M32). In this case, the supplementary information M2 and the image body M1 are decrypted using decryption methods which are appropriate for their respective encryption methods. Then, the CPU 11 reconstructs the medical image by attaching the decrypted supplementary information M2 to the image body M1 (which has been decrypted), to which an ID corresponding to the ID included in the supplementary information M2 has been attached (step S33), and stores the reconstructed medical image in the hard disk 12 (step S34), and the processing ends.

Here, since the volume of the supplementary information M2 is small compared to the volume of the image body M1, the time required for encryption processing can be reduced compared to the case of encrypting the medical image as is. Accordingly, even if the encryption is performed using a sophisticated encryption method, the processing time can be reduced compared to the case of encrypting the medical image as is. Therefore, the medical image can be sent and received at high speed while high security is maintained. Further, the medical image can be sent and received without using a high security circuit such as the dedicated circuit while high security is maintained.

Next, a third embodiment of the present invention will be described. In the third embodiment, the same reference numbers are assigned to the same elements with the configuration of the first embodiment, and detailed descriptions thereof are omitted here. The third embodiment differs from the first embodiment in that the supplementary information M2 is encrypted using higher security encryption method compared to the encryption method for encrypting the image body M1 and that the encrypted supplementary information M2 is sent using the dedicated circuit 4 and the encrypted image body M1 is sent using the public circuit 3.

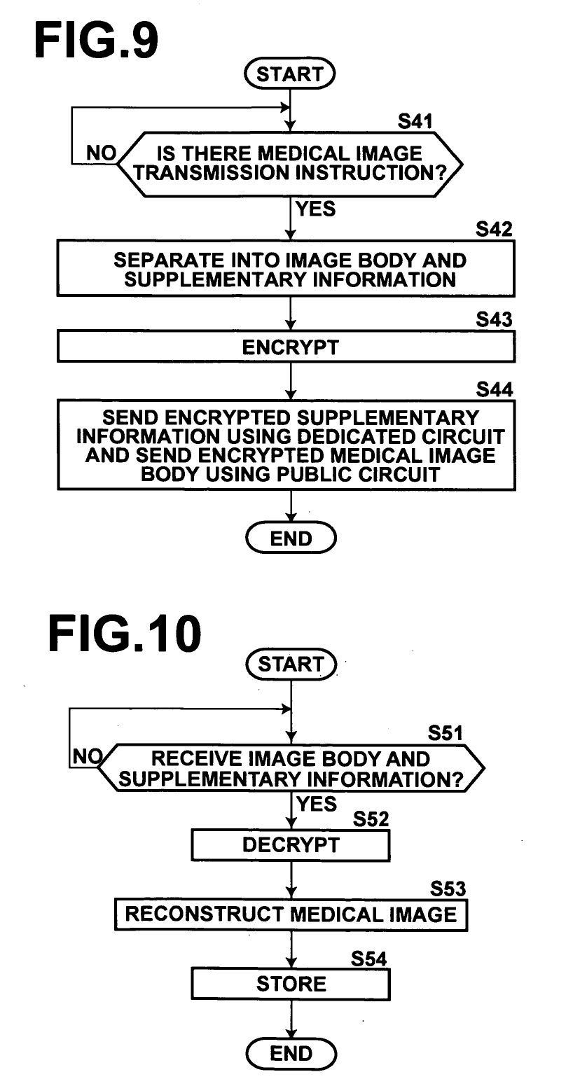

Next, processing performed in the third embodiment will be described. FIG. 9 shows a flowchart of processing performed at the time of sending the medical image from the personal computer 1A in the third embodiment. It is assumed that the medical image is stored in the hard disk 12 in the personal computer 1A. First, the CPU 11 in the personal computer 1A monitors whether the doctor who owns the personal computer 1A has given an instruction to send the medical image by using the input unit 16 (step S41). When step S41 is YES, the medical image of which transmission has been instructed is separated into the image body M1 and the supplementary information M2, as illustrated in FIG. 3B (step S42). In this case, the patient ID included in the supplementary information M2 is copied and attached to the image body M1.

Then, the image body M1 and the supplementary information M2 are encrypted (step S43). In this case, the supplementary information M2 is encrypted using a more sophisticated encryption method compared to the encryption method for encrypting the image body M1 so that the encrypted supplementary information M2 cannot easily be decrypted.

Next, the encrypted supplementary information M2 is sent to the personal computer 1B using the dedicated circuit 4, and the encrypted image body M1 is sent to the personal computer 1B using the public circuit 3 (step S44), and the processing ends.

FIG. 10 shows a flow chart of processing performed at the time of receiving the medical image by the personal computer 1B in the third embodiment. First, the CPU 11 in the personal computer 1B monitors whether the encrypted image body M1 and the encrypted supplementary information M2 have been received (step S51). When step S51 is YES, the CPU 11 decrypts the received image body M1 and the received supplementary information M2 (step M52). In this case, the supplementary information M2 and the image body M1 are decrypted using decryption methods which are appropriate for their respective encryption methods. Then, the CPU 11 reconstructs the medical image by attaching the decrypted supplementary information M2 to the image body M1 (which has been decrypted), to which an ID corresponding to the ID included in the supplementary information M2 has been attached (step S53), and stores the reconstructed medical image in the hard disk 12 (step S34), and the processing ends.

Here, since the volume of the supplementary information M2 is small compared to the volume of the image body M1, the time required for communication and the time required for encryption processing can be reduced compared to the case of sending and receiving the medical image as is. Consequently, the communication cost can be reduced compared to the case of sending and receiving the medical image as is even if a circuit such as the dedicated circuit 4, for which usage charges are expensive, is used. Further, the processing time can be reduced compared to the case of encrypting the whole medical image even if the encryption of the supplementary information is performed using the more sophisticated encryption method. Therefore, the medical image can be sent and received at low cost, while high security is maintained.

Next, a fourth embodiment of the present invention will be described. In the fourth embodiment, the same reference numbers are assigned to the same elements in the configuration of the first embodiment, and detailed descriptions thereof are omitted here. In the fourth embodiment, a first transmission method that encrypts the supplementary information M2 using a higher security encryption method compared to the encryption method for encrypting the image body M1 then sending the encrypted supplementary information M2 and the encrypted image body M1 using the public circuit 3 and a second transmission method for sending the supplementary information M2 using the dedicated circuit 4 and sending the image body M1 using the public circuit 3 can be selected. In this case, the input unit 16 functions as a selection accepting means.

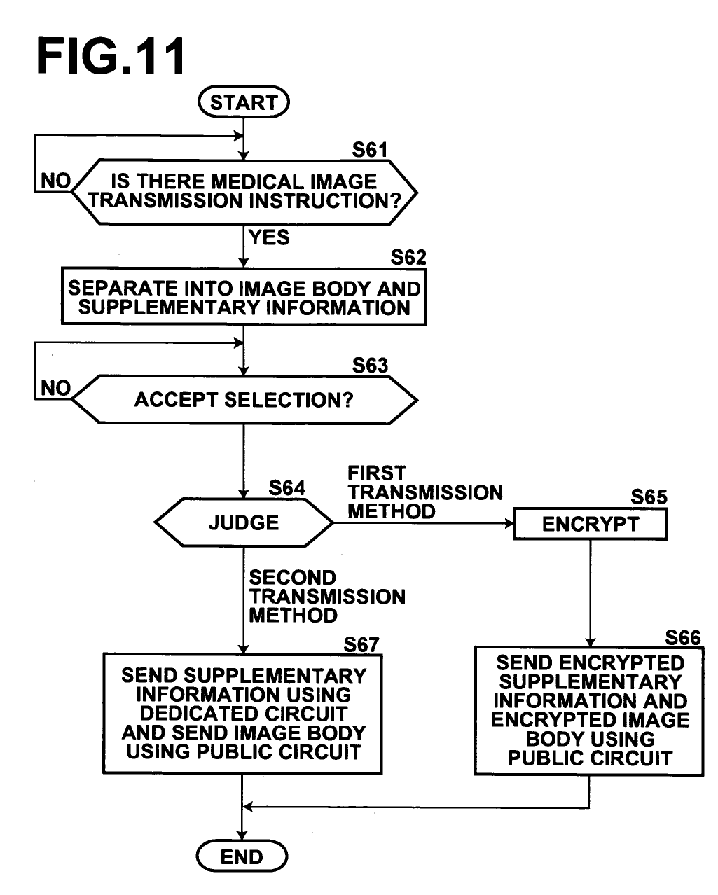

Next, processing performed in the fourth embodiment will be described. FIG. 11 shows a flow chart of processing performed at the time of sending the medical image from the personal computer 1A in the fourth embodiment. It is assumed that the medical image is stored in the hard disk 12 in the personal computer 1A. First, the CPU 11 in the personal computer 1A monitors whether the doctor who owns the personal computer 1A has given an instruction to send the medical image by using the input unit 16 (step S61). When step S61 is YES, the medical image, for which transmission has been instructed, is separated into the image body M1 and the supplementary information M2, as illustrated in FIG. 3B (step S62). In this case, the patient ID included in the supplementary information M2 is copied and attached to the image body M1.

Then, the CPU 11 starts to monitor whether a selection of the transmission method has been accepted from the input unit 16 (step S63). When step S63 is YES, a judgment is made on which transmission method has been selected from between the first transmission method and the second transmission method (step S64).

In the case that the first transmission method has been selected, the image body M1 and the supplementary information M2 are encrypted (step S65). In this case, the supplementary information M2 is encrypted using the more sophisticated encryption method compared to the encryption method for encrypting the image body M1, so that the encrypted supplementary information M2 cannot easily be decrypted. Next, the encrypted supplementary information M2 is sent to the personal computer 1B using the dedicated circuit 4 and the encrypted image body M1 is sent to the personal computer 1B using the public circuit 3 (step S65), and the processing ends.

On the other hand, in the case that the second transmission method has been selected, the supplementary information M2 is sent to the personal computer 1B using the dedicated circuit 4 and the image body M1 is sent to the personal computer 1B using the public circuit 3 (step S67), and the processing ends.

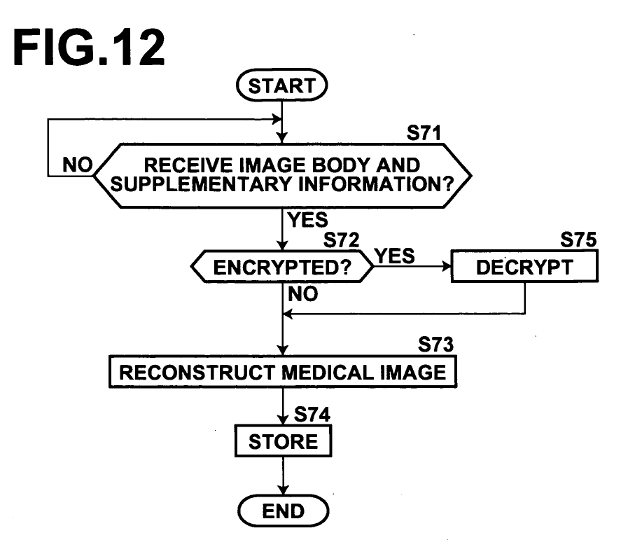

FIG. 12 shows a flow chart of processing performed at the time of receiving the medical image by the personal computer 1B in the fourth embodiment. First, the CPU 11 in the personal computer 1B monitors whether the image body M1 and the supplementary information M2 have been received (step S71). When step S71 is YES, a judgment is made on whether the received image body M1 and the received supplementary information M2 are encrypted (step S72). In the case that the received image body M1 and the received supplementary information M2 are not encrypted, step S72 is NO. Then, the CPU 11 reconstructs the medical image by attaching the supplementary information M2 to the image body M1 (which has been decrypted) to which an ID corresponding to the ID included in the supplementary information M2 has been attached (step S73) and stores the reconstructed medical image in the hard disk 12 (step S74), and the processing ends.

If step S72 is YES, the supplementary information M2 and the-image body M1 are decrypted (step S75), and the processing advances to step S73. Then, the medical image is reconstructed by attaching the supplementary information M2 to the image body to which an ID corresponding to the ID included in the supplementary information M2 has been attached (step S73), the reconstructed medical image is stored in the hard disk 12 (step S74), and the processing ends.

Further, in the above fourth embodiment, in the case that the encryption has been selected, both of the encrypted supplementary information M2 and the encrypted image body M1 may alternatively be sent using the public circuit 3.

Further, in the above first through fourth embodiments, the image body M1 and the supplementary information M2 are sent simultaneously. However, the image body M1 and the supplementary information M2 may alternatively be sent at different timings. Consequently, temporal series relationships between the image body M1 and the supplementary information M2 become reduced, and therefore, security at the time of sending the medical image can be improved.

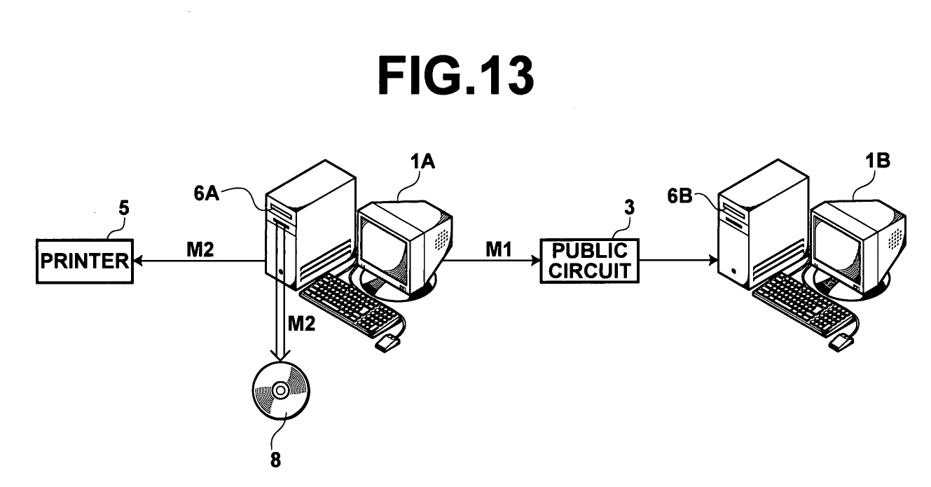

Next, a fifth embodiment of the present invention will be described. FIG. 13 is a schematic block diagram showing the configuration of a medical image sending and receiving system, to which an image sending device and an image receiving device according to the fifth embodiment of the present invention have been applied. As illustrated in FIG. 13, the medical image sending and receiving system according to the fifth embodiment is a system where the medical image is sent and received between the two personal computers 1A and 1B. The system is configured by connecting the personal computers 1A and 1B using the public circuit 3. Further, a printer 5 is connected to the personal computer 1A. Further, media drives 6A and 6B which record information in a medium M such as an FD, a CD-R, and an MO are provided in the bodies of the personal computers 1A and 1B.

In the fifth embodiment, the medical image is separated into the image body M1 and the supplementary information M2 at the personal computer 1A, and only the image body M1 is sent from the personal computer 1A to the personal computer 1B. Accordingly, leakage of the supplementary information via the communication circuit can be prevented.

The supplementary information M2 is stored in a medium 8 by using the media drive 6A, and the medium 8 is delivered to the doctor who owns the personal computer 1B to which the image body M1 has been sent. Further, the supplementary information M2 may alternatively be printed out from the printer 5 and delivered by facsimile, mail, or the like to a doctor who owns the terminal device to which the image body M1 has been sent. Here, as described above, since the patient ID included in the supplementary information M2 has been attached to the image body M1, even if the image body M1 and the supplementary information M2 are sent and delivered separately, the image body M1 and the supplementary information M2 can easily be associated with each other.

What is claimed is:

1. An image sending device having a sending means for sending a medical image, to which supplementary information including patient privacy information is attached, to an external device, comprising:
a separating means for separating the medical image into the supplementary information and an image body; and
a transmission control means for controlling the sending means so that the sending means sends the supplementary information using a higher security transmission method compared to a transmission method for sending the image body.

2. The image sending device as defined in claim 1, wherein the higher security transmission method is a transmission method that encrypts the supplementary information using a more sophisticated encryption method compared to an encryption method for encrypting the image body.

3. The image sending device as defined in claim 1, wherein the higher security transmission method is a transmission method for sending the supplementary information using a higher security communication circuit compared to a communication circuit for sending the image body.

4. The image sending device as defined in claim 1, further comprising:
a selection accepting means for accepting a selection of a first transmission method that encrypts the supplementary information using a more sophisticated encryption method compared to an encryption method for encrypting the image body and/or a second transmission method for sending the supplementary information using a higher security communication circuit compared to a communication circuit for sending the image body as the high security transmission method; wherein:
the transmission control means controls the sending means so that the sending means sends the supplementary information using the transmission method which has been accepted by the selection accepting means.

5. An image receiving device comprising:
a receiving means for receiving the supplementary information and the image body which have been sent by the image sending device as defined in claim 1; and
an associating means for associating the supplementary information with the image body.

6. An image sending device having a sending means for sending a medical image, to which supplementary information including patient privacy information is attached, to an external device, comprising:
a separating means for separating the medical image into the supplementary information and an image body; and
a transmission control means for controlling the sending means so that the sending means sends only the image body.

7. The image sending device as defined in claim 6, further comprising a recording means for recording the supplementary information in a recording medium.

8. The image sending device as defined in claim 6, further comprising:
a print-out means for printing out the supplementary information.
